(19) United States
(12) Patent Application Publication
(10) Pub. No.: US 2022/0054168 A1
(43) Pub. Date: Feb. 24, 2022

(54) DRIVER FOR ARTHROSCOPIC IMPLANT

(71) Applicant: Conmed Corporation, Largo, FL (US)

(72) Inventors: Ryo Isshiki, Largo, FL (US); Kenny Rush, Saint Petersburg, FL (US)

(73) Assignee: Conmed Corporation, Largo, FL (US)

(21) Appl. No.: 17/405,477

(22) Filed: Aug. 18, 2021

Related U.S. Application Data
(60) Provisional application No. 63/067,560, filed on Aug. 19, 2020.

(51) Int. Cl. A61B 17/34

(52) U.S. Cl. CPC A61B 17/3468; A61B 2017/00858

(57) ABSTRACT

A driver for a medical implant having a shaft with a longitudinal throughbore, a slot formed in the distal end of the shaft, and a rod moveable longitudinally within the shaft to selectively interlock the shaft with a medical implant or to release the medical implant once it is positioned in place during a medical operation. A pair of opposing ribs may extend circumferentially about the distal end of the shaft to assist with interlocking to the medical implant. The shaft of the driver expands radially when the rod is moved to the advanced position to interlock to the medical implant, and contracts radially when the rod is moved to the retracted position to release the medical implant. The shaft may include knurling to assist with engagement to the medical implant.

### CROSS-REFERENCE TO RELATED APPLICATIONS

The present application claims priority to U.S. Provisional App. No. 63/067,560, filed on Aug. 19, 2020, hereby incorporated by reference in its entirety.

### BACKGROUND OF THE INVENTION

### 1. Field of the Invention

The present invention relates to orthopedic instruments and, more particularly, to an orthopedic implant driver.

### 2. Description of the Related Art

Orthopedic implants may be used in arthroscopic procedures to support surgical repairs. In this scenario, the implant must be secured on an instrument referred to as a driver that can introduce the implant into the arthroscopic space and assist with implantation in the desired location. The interface between the driver and the implant must be secure enough ensure reliable introduction and implantation, but also allow reliable release of the implant from the driver upon completion of implantation so that the driver can be removed.

### BRIEF SUMMARY OF THE INVENTION

The present invention provides a secure approach for delivering a medical implant into a surgical location that also allow for easy release of the implant. A driver according to the present invention comprises a shaft extending longitudinally from a proximal end to a distal end and having a throughbore extending therethrough, a slot formed in a portion of the distal end of the shaft and in communication with the throughbore, and a rod positioned in the throughbore that is moveable longitudinally within the shaft between an advanced position, where the rod extends in the slot, and a retracted position, where the rod does not extend within the slot. A pair of opposing ribs may extend circumferentially about the distal end of the shaft. The shaft expands radially when the rod is moved from the retracted position to the advanced position. The shaft contracts radially when the rod is moved from the retracted position to the retracted position. The rod is moveable longitudinally within the shaft to extend from the distal end of the shaft. The distal end of the shaft may include knurling on both sides of the slot. A medical implant may be coupled to the shaft and secured in place by the rod in the advanced position. Movement of the rod into the retracted position will release the medical implant from the shaft. Movement of the rod into an extended position may activate a mechanism of the medical implant.

The present invention also comprises a method of providing a medical implant. A first step includes providing a driver having a shaft extending longitudinally from a proximal end to a distal end and having a throughbore extending therethrough, a slot formed in a portion of the distal end of the shaft and in communication with the throughbore, and a rod positioned in the throughbore and moveable longitudinally within the shaft between an advanced position, where the rod extends in the slot, and a retracted position, where the rod does not extend within the slot. Another step includes providing a medical implant having a bore that receives the distal end of the shaft and fixed thereto by the rod being positioned in the advanced position. A further step includes releasing the medical implant by moving the rod to the retracted position so that the shaft can exit the bore of the medical implant.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 is a side view of a driver according to the present invention with a rod in a withdrawn position;

FIG. 2 is a side view of a driver according to the present invention with a rod in an advanced position;

FIG. 3 is a cross-sectional view of an implant for use with a driver according to the present invention;

FIG. 4 is a cross-sectional view of a driver according to the present invention in combination with an implant;

FIG. 5 is a cross-sectional view of a driver according to the present interlocked with an implant;

FIG. 6 is a perspective view of alternate interlocking structures for a driver according to the present invention; and

FIG. 7 is a side view of FIG. 2 is a side view of a driver according to the present invention with a rod in an extended position.

### DETAILED DESCRIPTION OF THE INVENTION

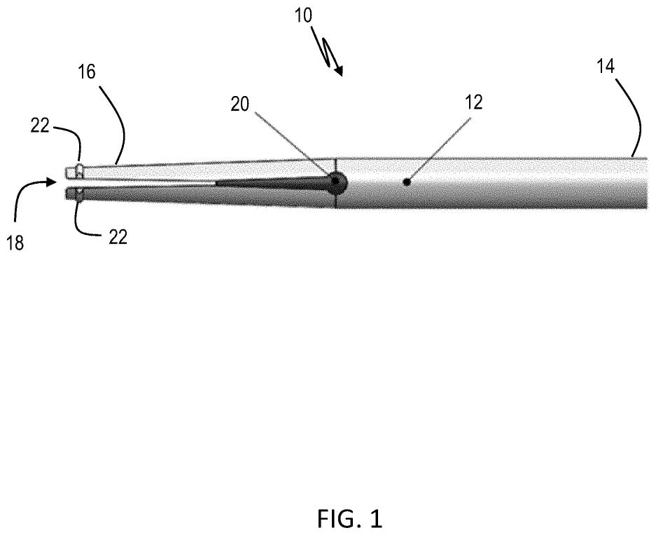

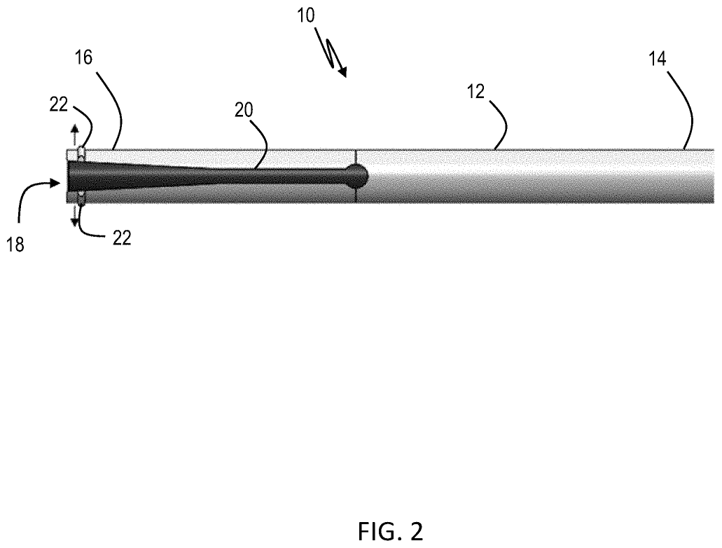

Referring the figures, wherein like numerals refer to like parts throughout, there is seen in FIG. 1 an orthopedic implant driver 10 comprising a tubular shaft 12 extending longitudinally from a proximal end 14 to a distal end 16 having a slot 18 formed therein that is in communication with the throughbore of shaft 12. As seen in FIG. 1, slot 18 generally bisects distal end 16 of shaft 12. A cylindrical rod 20 is positioned in shaft 12 and slidingly engaged therein for movement in the throughbore of shaft 12 between a retracted position as seen in FIG. 1, where rod 20 is withdrawn toward proximal end 14 of shaft 12, and an advanced position as seen in FIG. 2, where rod 20 extends within the portion of shaft 12 having slot 18. As further seen in FIGS. 1 and 2, distal end 16 of shaft 12 is tapered so that movement of rod 20 into the advanced position within slot 18 biases shaft 12 outwardly. As a result, shaft 12 is expanded radially when rod 20 is moved from the retracted position to the advanced position, and shaft 12 can contract radially when rod 20 is moved from the advanced position to the retracted position. Distal end 16 of shaft 12 may include a pair of opposing ribs 22 extending circumferentially around portions of distal end 16. Distal end 14 may comprise any variety of user grips, handles, etc. as is known in the art as well as a mechanism coupled to rod 20 to move rod 20 longitudinally within throughbore of shaft 12 including into the region of shaft 12 where slot 18 is formed, such as a lever mechanism coupled to the handle and rod 20.

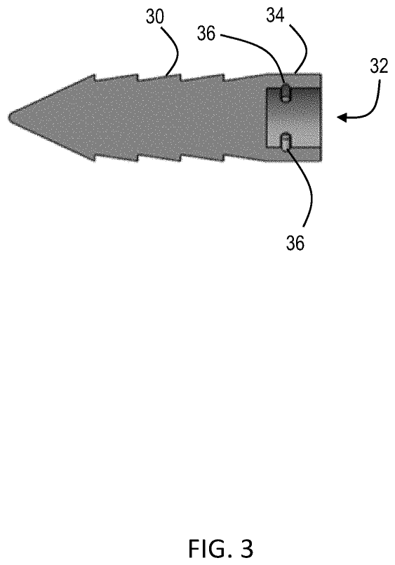

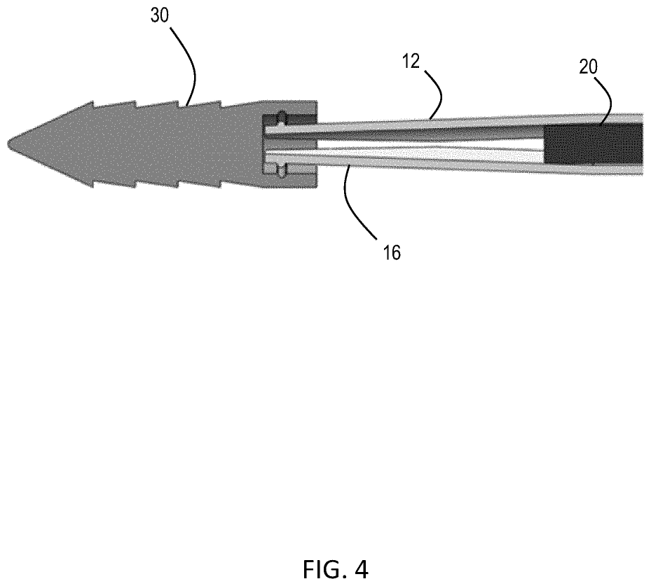

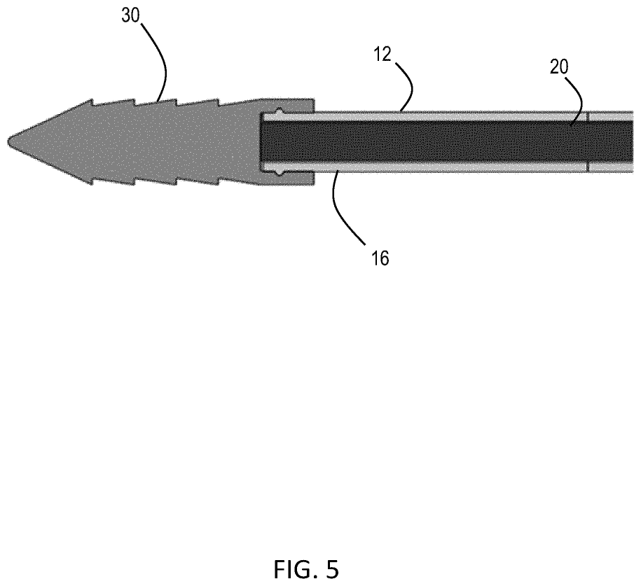

Referring to FIG. 3, an implant 30 to be inserted into a surgical location by implant driver includes a bore 32 formed in the proximal end 34 of implant 30. Bore 32 includes a pair of opposing notches 36 that corresponding in dimensions to pair of opposing ribs 22 of shaft 12 so that implant 30 and driver can be securely interlocked when rod 20 is in the advanced position, and released from engagement with each other when rod 20 is in the retracted position. As seen in FIG. 4, distal end 16 of shaft 12 and bore 32 are dimensioned to provide a clearance between shaft 12 and bore 32 with rod 20 in the retracted position so that shaft 12 may be inserted into bore 32 of implant 30. As seen in FIG. 5, advancement of rod 20 into the advanced position expands distal end 16 of shaft 12 so that ribs 22 engage notches 36 and the outer surface of shaft 12 frictionally engages the inner surface of bore 32. Driver 10 may now be used to location and insert implant 30 into a surgical location. Withdrawal of rod 20 into retracted position and out of distal end 16 of shaft 12 returns shaft 12 to the configuration of FIG. 4 so that driver 10 can be removed, thereby leaving implant 30 in place. As seen in FIGS. 1 and 2, shaft 12 is tapered in the location of slot 18 so that shaftl8 expands to be cylindrical along its length. Shaft 12 is preferably manufactured out of a material that will elastically deform in response to movement of rod 20, so that shaft 12 will return to the position seen in FIG. 1 when rod 20 is returned to the retracted position. For example, conventional surgical metals and biologically inert polymers may be used.

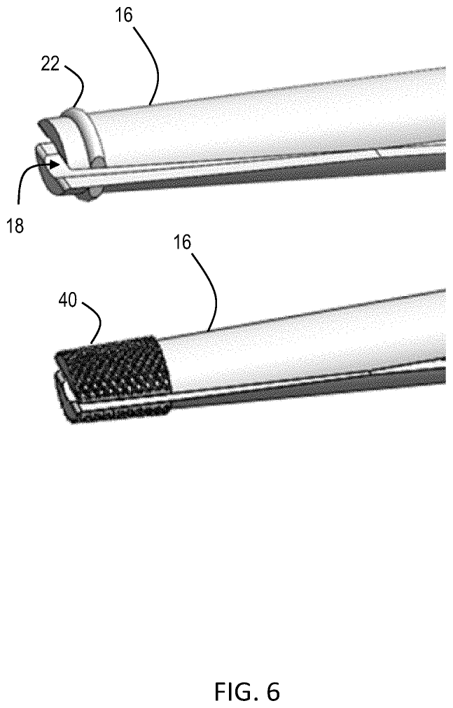

The interlocking between driver 10 and interlock 30 may be accomplished by pair of pair of opposing ribs 22 extending circumferentially around portions of distal end 16 as seen in FIGS. 1, 2 and 6. As further seen in FIG. 6, interlocking of driver 10 and medical device 30 may also be accomplished by other mechanical features that provide an interference fit between distal end 16 of shaft 12 and bore 32 of implant 30, such as a knurling 40 on distal end 16 of shaft 12 that will increase the frictional engagement between shaft 12 and implant 30. It should be recognized that other known mechanical engagement approaches may be used.

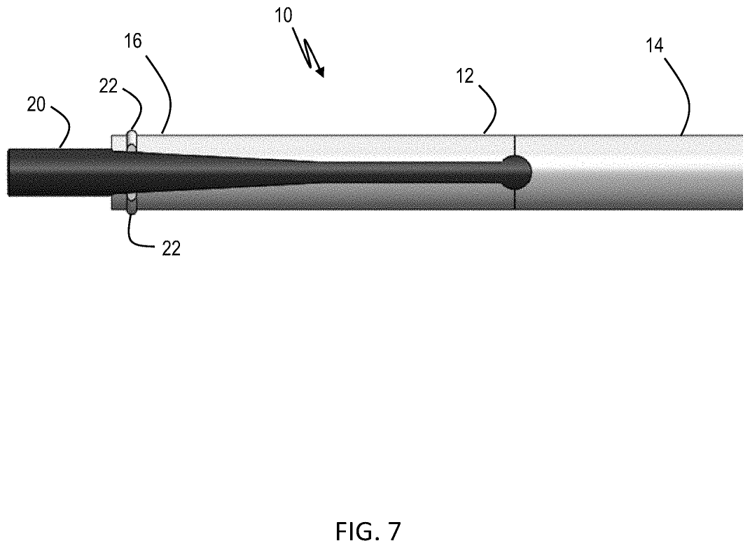

Referring to FIG. 7, rod 20 may be configured to move longitudinally within shaft to an extended position beyond the advance position, where rod 20 extends beyond distal end 16 of shaft 12. In this embodiment, rod 20 can be used to support or activate features of implant 30 that can be pushed or propped open in a specific configuration, or it may be used to actuate a mechanism within implant 30 that is distal to the interface holding implant 30 onto driver, to deploy those mechanisms once implant 30 is in place.

What is claimed is:

1. A driver for a medical implant, comprising:
a shaft extending longitudinally from a proximal end to a distal end and having a throughbore extending therethrough;
a slot formed in a portion of the distal end of the shaft and in communication with the throughbore; and
a rod positioned in the throughbore and moveable longitudinally within the shaft between an advanced position, where the rod extends in the slot, and a retracted position, where the rod does not extend within the slot.

2. The driver of claim 1, further comprising a pair of opposing ribs extending circumferentially about the distal end of the shaft.

3. The driver of claim 2, wherein the shaft will expand radially when the rod is moved from the retracted position to the advanced position.

4. The driver of claim 3, wherein the shaft will contract radially when the rod is moved from the advanced position to the retracted position.

5. The driver of claim 4, wherein the rod is moveable longitudinally within the shaft to extend from the distal end of the shaft.

6. The driver of claim 1, wherein the distal end of the shaft includes knurling on both sides of the slot.

7. The driver of claim 1, further comprising a medical implant coupled to the shaft and secured in place by the rod in the advanced position.

8. The driver of claim 7, wherein movement of the rod into the retracted position will release the medical implant from the shaft.

9. The driver of claim 8, wherein movement of the rod into an extended position activates a mechanism of the medical implant.

10. A method of providing a medical implant, comprising the steps of:
providing a driver having a shaft extending longitudinally from a proximal end to a distal end and having a throughbore extending therethrough, a slot formed in a portion of the distal end of the shaft and in communication with the throughbore, and a rod positioned in the throughbore and moveable longitudinally within the shaft between an advanced position, where the rod extends in the slot, and a retracted position, where the rod does not extend within the slot;
providing a medical implant having a bore that receives the distal end of the shaft and fixed thereto by the rod being positioned in the advanced position; and
releasing the medical implant by moving the rod to the retracted position so that the shaft can exit the bore of the medical implant.

11. The method of claim 10, wherein the shaft includes a pair of opposing ribs extending circumferentially about the distal end of the shaft.

12. The method of claim 11, wherein the shaft has expanded radially by the rod in the advanced position.

13. The method of claim 12, wherein the shaft contracts radially in response to the step of releasing the medical implant by moving the rod to the retracted position.

14. The method of claim 10, wherein the distal end of the shaft includes knurling on both sides of the slot.

15. The driver of claim 10, further comprising the step of moving the rod into an extended position to activate a mechanism of the medical implant.
